(19) United States
(12) Patent Application Publication
(10) Pub. No.: US 2023/0235280 A1
(43) Pub. Date: Jul. 27, 2023

(54) MODIFIED STEM CELLS AND METHODS OF USE THEREOF

(71) Applicant: New York Stem Cell Foundations, Inc., New York, NY (US)

(72) Inventors: Bjarki Johannesson, New York, NY (US); Deepta Bhattacharya, Tucson, AZ (US); Hannah Pizzato, Tucson, AZ (US)

(73) Assignee: Arizona Board of Regents on behalf of the University of Arizona, Tucson, AZ (US)

(21) Appl. No.: 17/768,146

(22) PCT Filed: Oct. 9, 2020
(86) PCT No.: PCT/US2020/055123

§ 371 Date: Apr. 11, 2022

(51) Int. Cl. C12N 5/0735; C12N 15/10; C12N 15/86

(52) U.S. Cl. CPC C12N 5/0606; C12N 15/102; C12N 15/86; C12N 2501/599; C12N 2506/02; C12N 2510/00; C12N 2750/14143

(57) ABSTRACT

The present invention provides modified stem cells (SCs) and use of the SCs to treat disease.

### CROSS-REFERENCE TO RELATED APPLICATIONS

This application claims benefit of priority under 35 U.S.C. § 119(e) of U.S. Provisional Patent Application Ser. No. 62/913,568, filed Oct. 10, 2019, the contents of which is incorporated herein by reference in its entirety.

### STATEMENT OF GOVERNMENT SUPPORT

This invention was made with government support under R21 AI132910 awarded by the National Institutes of Health. The government has certain rights in the invention.

### BACKGROUND INFORMATION

### Field of the Invention

The present invention relates generally to the field of medicine, and more specifically to genetically modified stem cells (SCs), such as genetically modified human embryonic stem cells (hESCs), and their use to treat disease.

### Background of the Invention

Regenerative medicine in the form of cell transplantation is one of the most promising therapeutic approaches for the treatment of intractable medical conditions such as diabetes, heart disease, and neurodegenerative diseases. However, a major hurdle toward implementing cell transplantation in the clinic is immune rejection of donor cells, especially when these are derived from a foreign host. While it is possible to address immune rejection, in part, by administering immunosuppressant drugs, these typically entail severe adverse side effects.

Organ transplantation provides an opportunity to treat people with certain diseases and can allow an organ recipient to live a full life. For example, in the case of end-stage liver, lung and heart disease, transplantation is generally the only available therapeutic option. There have been improvements in immunosuppressive drugs and ancillary care that have led to short-term patient and graft survival rates. However, this success is hampered by several problems, such as poor long-term graft survival rates, the need for continual immunosuppressive medication and the discrepancy between supply and demand of organs.

Allotransplantations have been developed to increase the supply of donor tissue. However, limiting the allogeneic response is a major challenge. Allogeneic transplants do not succeed unless the recipient's immune system, is downregulated. The current clinical standard is the use of systemic immunosuppressive medications, which reduce the efficacy of the graft and substantially increase the risk of infections.

Advances in understanding immune surveillance, as well as the ability to genetically modify cells, such as SCs, is allowing the generation of cells that avoid immune rejection. However, there is an ongoing need to develop improved technologies for cell transplantation therapies.

### SUMMARY OF THE INVENTION

The present invention provides genetically modified SCs, as well as methods for their generation and use to treat diseases, such as type 1 diabetes (T1D).

Accordingly, in embodiments, the invention provides a method of generating a genetically modified SC. In one aspect, the method includes: a) modifying a SC to reduce expression relative to a wild-type SC of HLA-I, HLA-II, or a combination thereof; and b) introducing exogenous constructs to express immune evasion genes comprising CR1 and/or CD24 and optionally one or more of CD47, CD55, CD46, CD59 and/or HLA-E-single chain trimer.

In some aspects, the immune evasion genes include CR1 and CD24 and optionally one or more of CD47, CD55, CD46, CD59 and/or HLA-E-single chain trimer.

In some aspects, the SCs may further be modified to express one or more of PDL1 and/or HLA-G-single chain trimer.

In some aspects, the immune evasion genes include CR1, CD24, CD47, CD55, CD46, CD59 and HLA-E-single chain trimer.

In another embodiment, the invention provides a genetically modified SC generated by the method of the invention.

In yet another embodiment, the invention provides a modified SC wherein: (i) expression of HLA-I and HLA-II is abrogated; and (ii) the SC is genetically modified to express CR1 and/or CD24 and optionally one or more of CD47, CD55, CD46, CD59 and/or HLA-E-single chain trimer.

In still another embodiment, the invention provides a cell line derived from a genetically modified SC of the invention.

In another embodiment, the invention provides a differentiated cell or tissue generated by differentiating a genetically modified SC of the invention. In various aspects, the cell or tissue is microglia, retinal pigmented epithelia, astrocytes, oligodendrocytes, hepatocytes, podocytes, keratinocytes, cardiomyocytes, dopaminergic neurons, cortical neurons, sensory neurons, NGN2-directed neurons, interneurons, basal forebrain cholinergic neurons, pancreatic beta cells, neural stem cells, natural killer cells, regulatory T cells, lung cell lineages, kidney cell lineages or blood cell lineages.

In another embodiment, the invention provides a β cell generated by differentiating a genetically modified SC of the invention.

In yet another embodiment, the invention provides a method of treating a disease or disorder in a subject in need thereof with a genetically modified SC, or progeny of a genetically modified SC of the invention.

In another embodiment, the invention provides a method of treating T1D in a subject by administering a genetically modified SC or β cell of the present invention to the subject, thereby treating T1D in the subject.

### BRIEF DESCRIPTION OF THE FIGURES

FIG. 1A depicts generation of an HLA-deficient hES cell line. Shown is a genome editing workflow. Cas9 and three gRNAs targeted to genes essential for HLA expression were nucleofected into H1 hES cells. Two rounds of subcloning and MiSeq™ analysis yield clonal mutant cell lines.

FIG. 1B depicts generation of an HLA-deficient hES cell line. Shown is an example of MiSeq™ analysis of targeted genes. Frameshift mutations were introduced in 5 of 6 alleles.

FIG. 1C depicts generation of an HLA-deficient hES cell line. Graphical data is shown of WT or HLA-KO hES cells that were stained for HLA-I expression with or without IFNg-treatment. HLA-I expression was absent in b2m- and TAP1-deficient cells.

FIG. 2A illustrates data showing that cord blood-humanized mice fail to reject xenogeneic teratomas. Shown is data representative of splenic chimerism of NSG-W41 mice 20 weeks after transplantation of cord blood CD34+ cells.

FIG. 2B illustrates data showing that cord blood-humanized mice fail to reject xenogeneic teratomas. Graphical data is shown of teratoma growth in humanized or control NSG-41 recipients following transplantation of unmodified or HM-KO hES cells.

FIG. 3 is a graphical illustration showing that expressing immune evasion genes allows teratoma growth in immune-competent mice. HLAI/IIKO hES cells were lentivirally transduced with the listed mouse immune evasion genes. Approximately 30% of cells were infected with any given lentivirus, leading to a relatively low frequency of cells expressing all 4 genes. These or control cells were transplanted in bulk into 5 WT C57B16/N mice and teratoma growth was measured over 8 weeks. Only cells receiving lentiviruses demonstrated growth.

FIG. 4 is a series of graphs related to selection of HM-KO cells expressing immune evasion genes. HM-KO cells were first transduced with lentiviruses encoding Crry, mCD55, mCD59, and K^{b}-single chain trimer. Cells were next sorted such that they uniformly expressed Crry, mCD59, and K^{b}-single chain trimer. Approximately 60% of these cells also expressed mCD55. These cells were used to generate β cells for xenotransplantation (left panel, pre-sort). These cells were grown, further transduced with mCD47, and then sorted for mCD55 expression ±CD47. These are the next generation of cells that will be used for xenotransplants. Pre- and post-sort flow cytometric profiles are shown.

FIG. 5 is a graph depicting the differentiation efficiency of WT, HM-KO and HM-KO-Lenti ECSs as measured by NKX6.1 expression levels at stages 4 and 7 of the differentiation protocol used in the Examples.

FIG. 6A is a series of images showing that HM-KO-Lenti stem cell-derived pseudo-islet grafts survive in immunocompetent mice 1 week following transplantations, as shown by GFP IHC.

FIG. 6B is a series of images showing that HM-KO-Lenti stem cell-derived pseudo-islet grafts survive in immunocompetent mice 2 months following transplantations, as shown by GFP IHC.

FIG. 7 are images of native mammary glands from the same section shown in FIGS. 6A and 6iB. The absence of GFP highlights specificity of the signal in FIGS. 6A and 6B.

FIG. 8 is a graph showing human C-peptide blood levels in mice transplanted with genetically modified pseudo-islets.

FIG. 9A is an image showing correction of AAVS targeting. Shown is a depiction of the locus that all current AAVS targeting vectors are designed to target, relative to the actual site of adeno-associated virus integration.

FIG. 9B is an image showing correction of AAVS targeting. A schematic is shown of an example modified vector designed to target mouse immune evasion genes to the actual AAV integration site. To further minimize the chances of silencing, an upstream chromatin opening element was included upstream of the hEF1a promoter.

FIG. 9C is an image showing correction of AAVS targeting. Shown is data relating to HM-KO or HUES2 cells that were transfected with Cas9 and gRNAS along with the original or modified AAVS targeting constructs encoding mCD59, mCrry, mQa1-SCT, and neomycin resistance. Cells were selected for 2 weeks in neomycin, and drug resistant cells were analyzed for mCD59 expression.

FIG. 9D is an image showing correct targeting of the AAVS locus. Data is shown of cells positive for mCD59 that were single cell sorted for expansion. Reanalysis was performed >8 weeks in culture.

FIG. 10A is an image showing that AAVS constructs mediate immune evasion. Data is shown of CHO cells that were transfected with an AAVS targeting construct expressing human CD55, CD46, and HLA-E. Transfectants were stained with a-CHO antibody and then with C7-deficient human serum. Cells were tested for C3c, C3d, and C4c complement deposition.

FIG. 10B is an image showing that AAVS constructs mediate immune evasion. Data is shown of 721.221 cells that were transfected with the same construct as in FIG. 10A which were cultured with primary human NK cells. NK cell degranulation was measured as a function of CD107a expression.

FIG. 11A depicts data showing improvements to a β cell differentiation protocol made through multiple rounds of Design of Experiment (DoE) optimizations.

FIG. 11B is an image showing improvements to a β cell differentiation protocol made through multiple rounds of DoE optimizations.

FIG. 11C depicts data showing improvements to a β cell differentiation protocol made through multiple rounds of DoE optimizations.

FIG. 12 is an image showing an experimental workflow to determine an optimal combination of evasion gene constructs, perform experiments in WT and NOD mice as well as generate a HM-KO cell line that stably expresses selected evasion gene constructs through AAVS targeting.

### DETAILED DESCRIPTION OF THE INVENTION

The present invention is based on the discovery of immune evasion factors that may be used to generated modified SCs useful for treatment of disease.

Before the present compositions and methods are described, it is to be understood that this invention is not limited to the particular cells, methods and/or experimental conditions described herein, as such cells, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the cell” include one or more cells and references to “the method” include one or more methods, and/or steps of the type described herein which will become apparent to those persons skilled in the art upon reading this disclosure and so forth.

The present invention is based, at least in part, on the discovery of immune evasion factors that may be used to generated modified SCs. In some aspects, the invention relies on genetically engineering an SC to include mutations in genes (for example, using CRISPR/Cas9) that result in a substantially non-immunogenic or minimally immunogenic SC for transplantation, as well as, express genes that prevent complement deposition to eliminate major determinants of immunogenicity. The modified SCs of the present invention provide scalable off-the-shelf therapies for treatment of a host of diseases, such as autoimmune disorders, neurodegenerative diseases, cancer, and infectious disease, as well as, the general application of SC based therapeutics using cells altered to avoid immune rejection.

Accordingly, in embodiments, the invention provides a method of generating a genetically modified SC. The method includes: a) modifying a SC to reduce expression relative to a wild-type SC of HLA-I, HLA-II, or a combination thereof, and b) introducing exogenous constructs to express immune evasion genes comprising CR1 and/or CD24 and optionally one or more of CD47, CD55, CD46, CD59 and/or HLA-E-single chain trimer.

In a related embodiment, the invention provides a modified SC wherein: (i) expression of HLA-I and HLA-II is abrogated; and (ii) the SC is genetically modified to express CR1 and/or CD24 and optionally one or more of CD47, CD55, CD46, CD59 and/or HLA-E-single chain trimer.

As described herein, methods and targets used to modify human SCs so that they evade recognition of several arms of the immune system have been developed. The present disclosure provides methods to generate a minimally immunogenic donor SC line that can be used without host immunosuppression for regenerative medicine therapies, as well as, cells generated by such methods.

Described herein are substantially or minimally immunogenic SCs (for example, hESCs) for transplantation, in particular, SC-based immunotherapies for various diseases. The creation of such cells and cell lines for transplantation allows for scalable off-the-shelf cellular therapies. This is desirable for most SC-based therapies being developed by private industry. Such cells can also facilitate regenerative medicine treatments for tissues destroyed by autoimmunity, such as pancreatic β cells in T1D and oligodendrocytes in multiple sclerosis.

As discussed herein and illustrated in the Examples, an SC was genetically modified such that the cell evades recognition by several arms of the immune system. SCs containing the modifications described in the present invention, alone or in combination with those previously described, can evade recognition by CD8+ T cells, CD4+ T cells, NK cells, complement, or phagocytotic cells. Furthermore, the cells can contain inducible suicide genes and drug resistance cassettes. This allows for selective elimination of grafts in case of adverse effects, and facile drug selection in culture to identify clonal cell lines. Together the process allows for the generation of SCs with significantly reduced immunogenicity for transplantation.

Disrupting specific immune receptors and introducing specific transgenes into SCs (e.g., modified by gene deletions and/or transgene (cDNA) insertions) can result in a universal donor SC. In some aspects, provided herein is a genetically engineered SC wherein HLA-I expression is reduced or eliminated to prevent direct recognition by allogeneic CD8+ T cells; and/or HLA-II expression is eliminated thus evading direct recognition by CD4+ T cells; and/or NKG2D ligand encoding genes are genetically modified to evade NK cell recognition.

In some aspects, β2 microglobulin and/or TAP1 encoding genes are genetically modified to inhibit or eliminate HLA-I expression.

In some aspects, CD74 and/or CIITA encoding genes are genetically modified to inhibit or eliminate HLA-II expression.

In some aspects, MICA and/or MICB encoding genes are genetically modified to evade NK cell recognition.

In some aspects, β2 microglobulin, TAP1 and CD74 are genetically modified to inhibit or eliminate HLA-I and HLA-II expression.

In some aspects, β2 microglobulin, TAP1, CD74 and CIITA are genetically modified to inhibit or eliminate HLA-I and HLA-II expression.

In some aspects, the NKG2D ligand encoding genes that are genetically modified to evade NK cell recognition include one or more of MICA, MICB, Raet1e, Raet1g, Raet11, Ulbp1, Ulbp2, and/or Ulbp3. In some aspects, the NKG2D ligand encoding gene that is genetically modified is MICA or MICB; or MICA in combination with MICB.

Also provided herein is a method for making a genetically engineered SC including delivering a construct to an AAVS locus in the SC to express one or more of the following genes (or immune evasion factor): CR1, CD24, CD47, CD55, CD46, CD59, HLA-E-single chain trimer, PDL1 and/or HLA-G-single chain trimer. It will be understood that one or more constructs may be utilized such that expression of any combination of the genes is achieved in the SC. In some aspects, the construct(s) may be designed to express CR1 and/or CD24. In one aspect the construct(s) may be designed to express CR1 and CD47. In one aspect the construct(s) may be designed to express CD24, CD46, CD55 and CD59. In one aspect the construct(s) may be designed to express CR1, CD24, HLA-E-single chain trimer, PDL1 and HLA-G-single chain trimer. In one aspect the construct(s) may be designed to express CR1, CD24, CD47, CD55, CD46, CD59, HLA-E-single chain trimer and PDL1. In one aspect the construct(s) may be designed to express CR1, CD47, HLA-E-single chain trimer and PDL1. In one aspect the construct(s) may be designed to express CD24, CD46, CD55, CD59, HLA-E-single chain trimer and PDL1. In one aspect the construct(s) may be designed to express CR1, CD24, CD47, CD55, CD46, CD59, HLA-E-single chain trimer, PDL1 and HLA-G-single chain trimer. In one aspect the construct(s) may be designed to express CR1 and/or CD24 and optionally one or more of CD47, CD55, CD46, CD59 and/or HLA-E-single chain trimer. In one aspect the construct(s) may be designed to express CR1 and/or CD24 and optionally one or more of CD47, CD55, CD46, CD59, HLA-E-single chain trimer, PDL1 and/or HLA-G-single chain trimer.

In some aspects, the present invention provides for genetic modifications in the 32 microglobulin and TAP1-encoding genes. This eliminates HLA-I expression and prevents direct recognition by allogeneic CD8+ T cells. As described herein the genetic modification can be an inactivating mutation.

In some aspects, the present invention further provides for mutations in genes encoding CD74 and optionally CIITA. This eliminates HLA-II expression and evades direct recognition by CD4+ T cells.

As described herein, an inactivating mutation can be any mutation in a gene resulting in reduction or elimination of expression of HLA-I or HLA-II. Inactivating mutations can include nucleotide insertions or deletions that change the reading frame and prevent translation of a functional protein.

The Examples of the present disclosure further show that these HLA-deficient cells generate teratomas in xenochimeric mice reconstituted with an allogeneic human immune system. As shown herein, it has been demonstrated that the cells lack expression of HLA-I and HLA-II. The disclosure further demonstrates that the AAVS targeting constructs properly express all intended genes and confer resistance to natural killer cell recognition and complement deposition.

The present invention further provides for the design of and validation of constructs to be delivered to the AAVS locus in SCs. These constructs encode genes that lead to evasion of NK cell recognition and phagocytosis. Expression of these genes substantially reduces NK cell activation. These constructs simultaneously encode inducible suicide genes and drug resistance cassettes. This allows for selective elimination of grafts in case of adverse effects, and facile drug selection in culture to identify clonal cell lines. Together the process allows for the generation of human pluripotent stem cells with significantly reduced immunogenicity for transplantation.

The present invention further provides for the design of and validation of constructs to be delivered to the AAVS locus in SC cells that lead to evasion of complement fixation.

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus or activating agent (e.g., a doxycycline- or tetracycline-inducible promoter). A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (e.g., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine), Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, Tex.; Sigma Aldrich, Mo.; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinofrmatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

In various aspects, the modified SC of the present invention is an induced pluripotent SCs (iPSCs) or embryonic SCs. In various aspects, the SC is mammalian, for example, human or mouse. In one aspect, the SC is derived from the subject to be treated. For example, a somatic cell may be harvested from a subject and reprogrammed to produce an iPSC which is then modified using the method of the present invention.

As used herein “adult” means post-fetal, e.g., an organism from the neonate stage through the end of life, and includes, for example, cells obtained from delivered placenta tissue, amniotic fluid and/or cord blood.

As used herein, the term “adult differentiated cell” encompasses a wide range of differentiated cell types obtained from an adult organism, that are amenable to producing iPSCs using the instantly described automation system. Preferably, the adult differentiated cell is a “fibroblast.” Fibroblasts, also referred to as “fibrocytes” in their less active form, are derived from mesenchyme. Their function includes secreting the precursors of extracellular matrix components including, e.g., collagen. Histologically, fibroblasts are highly branched cells, but fibrocytes are generally smaller and are often described as spindle-shaped. Fibroblasts and fibrocytes derived from any tissue may be employed as a starting material for the automated workflow system on the invention.

As used herein, the term, “induced pluripotent stem cells” or, iPSCs, means that the stem cells are produced from differentiated adult cells that have been induced or changed, e.g., reprogrammed into cells capable of differentiating into tissues of all three germ or dermal layers: mesoderm, endoderm, and ectoderm. The iPSCs produced do not refer to cells as they are found in nature.

The terms “stem cell” or “undifferentiated cell” as used herein, refer to a cell in an undifferentiated or partially differentiated state that has the property of self-renewal and has the developmental potential to differentiate into multiple cell types, without a specific implied meaning regarding developmental potential (e.g., totipotent, pluripotent, and multipotent). A stem cell is capable of proliferation and giving rise to more such stem cells while maintaining its developmental potential. In theory, self-renewal can occur by either of two major mechanisms. Stem cells can divide asymmetrically, which is known as obligatory asymmetrical differentiation, with one daughter cell retaining the developmental potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. While each of these multipotent cells may be considered stem cells, the range of cell types each such stem cell can give rise to, e.g., their developmental potential, can vary considerably. Alternatively, some of the stem cells in a population can divide symmetrically into two stem cells, known as stochastic differentiation, thus maintaining some stem cells in the population as a whole, while other cells in the population give rise to differentiated progeny only. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

The term “differentiated cell” encompasses any somatic cell that is not, in its native form, pluripotent, as that term is defined herein. Thus, the term a “differentiated cell” also encompasses cells that are partially differentiated, such as multipotent cells, or cells that are stable, non-pluripotent partially reprogrammed, or partially differentiated cells, generated using any of the compositions and methods described herein. In some embodiments, a differentiated cell is a cell that is a stable intermediate cell, such as a non-pluripotent, partially reprogrammed cell. The transition of a differentiated cell (including stable, non-pluripotent partially reprogrammed cell intermediates) to pluripotency requires a reprogramming stimulus beyond the stimuli that lead to partial loss of differentiated character upon placement in culture. Reprogrammed and, in some embodiments, partially reprogrammed cells, also have the characteristic of having the capacity to undergo extended passaging without loss of growth potential, relative to parental cells having lower developmental potential, which generally have capacity for only a limited number of divisions in culture. In some embodiments, the term “differentiated cell” also refers to a cell of a more specialized cell type (e.g., decreased developmental potential) derived from a cell of a less specialized cell type (e.g., increased developmental potential) (e.g., from an undifferentiated cell or a reprogrammed cell) where the cell has undergone a cellular differentiation process.

The term “reprogramming” as used herein refers to a process that reverses the developmental potential of a cell or population of cells (e.g., a somatic cell). Stated another way, reprogramming refers to a process of driving a cell to a state with higher developmental potential, e.g., backwards to a less differentiated state. The cell to be reprogrammed can be either partially or terminally differentiated prior to reprogramming. In some embodiments of the aspects described herein, reprogramming encompasses a complete or partial reversion of the differentiation state, e.g., an increase in the developmental potential of a cell, to that of a cell having a pluripotent state. In some embodiments, reprogramming encompasses driving a somatic cell to a pluripotent state, such that the cell has the developmental potential of an embryonic stem cell, e.g., an embryonic stem cell phenotype. In some embodiments, reprogramming also encompasses a partial reversion of the differentiation state or a partial increase of the developmental potential of a cell, such as a somatic cell or a unipotent cell, to a multipotent state. Reprogramming also encompasses partial reversion of the differentiation state of a cell to a state that renders the cell more susceptible to complete reprogramming to a pluripotent state when subjected to additional manipulations, such as those described herein. Such manipulations can result in endogenous expression of particular genes by the cells, or by the progeny of the cells, the expression of which contributes to or maintains the reprogramming. In certain embodiments, reprogramming of a cell using the synthetic, modified RNAs and methods thereof described herein causes the cell to assume a multipotent state (e.g., is a multipotent cell). In some embodiments, reprogramming of a cell (e.g., a somatic cell) using the synthetic, modified RNAs and methods thereof described herein causes the cell to assume a pluripotent-like state or an embryonic stem cell phenotype. The resulting cells are referred to herein as “reprogrammed cells,” “somatic pluripotent cells,” and “RNA-induced somatic pluripotent cells.” The term “partially reprogrammed somatic cell” as referred to herein refers to a cell which has been reprogrammed from a cell with lower developmental potential by the methods as disclosed herein, such that the partially reprogrammed cell has not been completely reprogrammed to a pluripotent state but rather to a non-pluripotent, stable intermediate state. Such a partially reprogrammed cell can have a developmental potential lower that a pluripotent cell, but higher than a multipotent cell, as those terms are defined herein. A partially reprogrammed cell can, for example, differentiate into one or two of the three germ layers, but cannot differentiate into all three of the germ layers.

The term a “reprogramming factor,” as used herein, refers to a developmental potential altering factor, as that term is defined herein, such as a gene, protein, RNA, DNA, or small molecule, the expression of which contributes to the reprogramming of a cell, e.g., a somatic cell, to a less differentiated or undifferentiated state, e.g., to a cell of a pluripotent state or partially pluripotent state. A reprogramming factor can be, for example, transcription factors that can reprogram cells to a pluripotent state, such as SOX2, OCT3/4, KLF4, NANOG, LIN-28, c-MYC, and the like, including as any gene, protein, RNA or small molecule, that can substitute for one or more of these in a method of reprogramming cells in vitro. In some embodiments, exogenous expression of a reprogramming factor, using the synthetic modified RNAs and methods thereof described herein, induces endogenous expression of one or more reprogramming factors, such that exogenous expression of one or more reprogramming factors is no longer required for stable maintenance of the cell in the reprogrammed or partially reprogrammed state.

As used herein, the term “differentiation factor” refers to a developmental potential altering factor, as that term is defined herein, such as a protein, RNA, or small molecule, which induces a cell to differentiate to a desired cell-type, e.g., a differentiation factor reduces the developmental potential of a cell. In some embodiments, a differentiation factor can be a cell-type specific polypeptide, however this is not required. Differentiation to a specific cell type can require simultaneous and/or successive expression of more than one differentiation factor. In some aspects described herein, the developmental potential of a cell or population of cells is first increased via reprogramming or partial reprogramming using synthetic, modified RNAs, as described herein, and then the cell or progeny cells thereof produced by such reprogramming are induced to undergo differentiation by contacting with, or introducing, one or more synthetic, modified RNAs encoding differentiation factors, such that the cell or progeny cells thereof have decreased developmental potential.

In the context of cell ontogeny, the term “differentiate”, or “differentiating” is a relative term that refers to a developmental process by which a cell has progressed further down a developmental pathway than its immediate precursor cell. Thus in some embodiments, a reprogrammed cell as the term is defined herein, can differentiate to a lineage-restricted precursor cell (such as a mesodermal stem cell), which in turn can differentiate into other types of precursor cells further down the pathway (such as a tissue specific precursor, for example, a cardiomyocyte precursor), and then to an end-stage differentiated cell, which plays a characteristic role in a certain tissue type, and may or may not retain the capacity to proliferate further.

Therapeutic Applications and Formulations

As discussed herein, the invention further provides a method of treating a disease or disorder in a subject. In some aspects, the method includes administering to the subject a genetically modified SC of the present invention. In some aspects, the method includes administering a progeny of a genetically modified SC of the invention, such as a partially or terminally differentiated cell or tissue. In one aspect, the progeny is a progenitor cell generated from an SC of the present invention. A progenitor cell is a biological cell that, like an SC, has a tendency to differentiate into a specific type of cell, but is already more specific than a SC and is pushed to differentiate into its “target” cell. For example, a progenitor cell can be a hemogenic progenitor cell (e.g., hemogenic endothelial cell) or hematopoietic progenitor cell.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities. Thus, in addition to the therapies described herein, one may also provide to the subject other therapies known to be efficacious for treatment of the disease, disorder, or condition.

As discussed herein, the invention provides a process of treating a disease (e.g., an autoimmune disease, a tissue destroyed by an autoimmune disease, a pathogen, cancer, enzyme deficiency, or a neurodegenerative disease) with a cell-based therapy (e.g., differentiated progeny of a genetically engineered stem cell) in a subject in need thereof and administration of a therapeutically effective amount of a cell-based therapy, so as to treat the disease with an SC or progeny thereof while evading natural killer cell recognition.

Further, SCs of the present invention modified to avoid immune rejection using the methods of the present invention can be used to generate any other cell type currently being developed for use in patient therapy. Such differentiated cells are administered to a patient in need of such cells with reduced or without the need for immune suppressive agents.

Methods described herein are generally performed on a subject in need thereof. A subject in need of the therapeutic methods described herein can be a subject having, diagnosed with, suspected of having, or at risk for developing a disease. The subject can be an animal subject, including a mammal, such as horses, cows, dogs, cats, sheep, pigs, mice, rats, monkeys, hamsters, guinea pigs, and chickens, and humans. For example, the subject can be a human subject.

According to the methods described herein, administration can be parenteral, pulmonary, oral, topical, intradermal, ossicle, intramuscular, intraperitoneal, intravenous, subcutaneous, intranasal, epidural, ophthalmic, buccal, or rectal administration.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4.sup.th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

In some aspects, the cells, tissues, compositions and methods can be used to treat a neurodegenerative disease or disorder. For example, the neurodegenerative disease or disorder can be Alzheimer's disease, amyotrophic lateral sclerosis (ALS), Alexander disease, Alpers' disease, Alpers-Huttenlocher syndrome, alpha-methylacyl-CoA racemase deficiency, Andermann syndrome, Arts syndrome, ataxia neuropathy spectrum, ataxia (e.g., with oculomotor apraxia, autosomal dominant cerebellar ataxia, deafness, and narcolepsy), autosomal recessive spastic ataxia of Charlevoix-Saguenay, Batten disease, beta-propeller protein-associated neurodegeneration, Cerebro-Oculo-Facio-Skeletal Syndrome (COFS), Corticobasal Degeneration, CLN1 disease, CLN10 disease, CLN2 disease, CLN3 disease, CLN4 disease, CLN6 disease, CLN7 disease, CLN8 disease, cognitive dysfunction, congenital insensitivity to pain with anhidrosis, dementia, familial encephalopathy with neuroserpin inclusion bodies, familial British dementia, familial Danish dementia, fatty acid hydroxylase-associated neurodegeneration, Gerstmann-Straussler-Scheinker Disease, GM2-gangliosidosis (e.g., AB variant), HMSN type 7 (e.g., with retinitis pigmentosa), Huntington's disease, infantile neuroaxonal dystrophy, infantile-onset ascending hereditary spastic paralysis, Huntington's disease (HD), infantile-onset spinocerebellar ataxia, juvenile primary lateral sclerosis, Kennedy's disease, Kuru, Leigh's Disease, Marinesco-Sjogren syndrome, Mild Cognitive Impairment (MCI), mitochondrial membrane protein-associated neurodegeneration, Motor neuron disease, Monomelic Amyotrophy, Motor neuron diseases (MND), Multiple System Atrophy, Multiple System Atrophy with Orthostatic Hypotension (Shy-Drager Syndrome), multiple sclerosis, multiple system atrophy, neurodegeneration in Down's syndrome (NDS), neurodegeneration of aging, Neurodegeneration with brain iron accumulation, neuromyelitis optica, pantothenate kinase-associated neurodegeneration, Opsoclonus Myoclonus, prion disease, Progressive Multifocal Leukoencephalopathy, Parkinson's disease (PD), PD-related disorders, polycystic lipomembranous osteodysplasia with sclerosing leukoencephalopathy, prion disease, progressive external ophthalmoplegia, riboflavin transporter deficiency neuronopathy, Sandhoff disease, Spinal muscular atrophy (SMA), Spinocerebellar ataxia (SCA), Striatonigral degeneration, Transmissible Spongiform Encephalopathies (Prion Diseases), or Wallerian-like degeneration.

In some aspects, the cells, tissues, compositions and methods can be used treat cancer. For example, the cancer can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer (Head and Neck Cancer); Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer (Head and Neck Cancer); Leukemia; Lip and Oral Cavity Cancer (Head and Neck Cancer); Liver Cancer; Lung Cancer (Non-Small Cell and Small Cell); Lymphoma; Male Breast Cancer; Malignant Fibrous Histiocytoma of Bone or Osteosarcoma; Melanoma; Intraocular (Eye); Merkel Cell Carcinoma (Skin Cancer); Mesothelioma, Malignant; Metastatic Cancer; Metastatic Squamous Neck Cancer with Occult Primary (Head and Neck Cancer); Midline Tract Carcinoma Involving NUT Gene; Mouth Cancer (Head and Neck Cancer); Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer (Head and Neck Cancer); Nasopharyngeal Cancer (Head and Neck Cancer); Neuroblastoma; Non-Hodgkin Lymphoma; Non-Small Cell Lung Cancer; Oral Cancer, Lip or Oral Cavity Cancer; Oropharyngeal Cancer (Head and Neck Cancer); Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer (Head and Neck Cancer); Parathyroid Cancer; Penile Cancer; Pharyngeal Cancer (Head and Neck Cancer); Pheochromocytoma; Pituitary Tumor; Plasma Cell Neoplasm/Multiple Myeloma; Pleuropulmonary Blastoma; Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer (Head and Neck Cancer); Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sezary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic (Head and Neck Cancer); Stomach (Gastric) Cancer; T-Cell Lymphoma, Cutaneous; Lymphoma; Mycosis Fungoides and Sezary Syndrome; Testicular Cancer; Throat Cancer (Head and Neck Cancer); Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer); Urethral Cancer; Uterine Cancer, Endometrial; Uterine Sarcoma; Vaginal Cancer; Vascular Tumors (Soft Tissue Sarcoma); Vulvar Cancer; or Wilms Tumor.

In some aspects, the cells, tissues, compositions and methods can be used to treat an autoimmune disease or disorder. For example, the autoimmune disease or disorder can be Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune angioedema; Autoimmune dysautonomia; Autoimmune encephalomyelitis; Autoimmune hepatitis; Autoimmune inner ear disease (AIED); Autoimmune myocarditis; Autoimmune oophoritis; Autoimmune orchitis; Autoimmune pancreatitis; Autoimmune retinopathy; Autoimmune urticaria; Axonal & neuronal neuropathy (AMAN); Bab disease; Behcet's disease; Benign mucosal pemphigoid; Bullous pemphigoid; Castleman disease (CD); Celiac disease; Chagas disease; Chronic inflammatory demyelinating polyneuropathy (CIDP); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease; Congenital heart block; Coxsackie myocarditis; CREST syndrome; Crohn's disease; Dermatitis herpetiformis; Dermatomyositis; Devic's disease (neuromyelitis optica); Discoid lupus; Dressler's syndrome; Endometriosis; Eosinophilic esophagitis (EoE); Eosinophilic fasciitis; Erythema nodosum, Essential mixed cryoglobulinemia; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inverse); Hypogammalglobulinemia, IgA Nephropathy; IgG4-related sclerosing disease; Immune thrombocytopenic purpura (ITP); Inclusion body myositis (IBM); Interstitial cystitis (IC); Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus, Ligneous conjunctivitis; Linear IgA disease (LAD); Lupus; Lyme disease chronic; Meniere's disease; Microscopic polyangiitis (MPA); Mixed connective tissue disease (MCTD); Mooren's ulcer; Mucha-Habermann disease; Multifocal Motor Neuropathy (MMN) or MMNCB, Multiple sclerosis; Myasthenia gravis; Myositis; Narcolepsy; Neonatal Lupus; Neuromyelitis optica; Neutropenia; Ocular cicatricial pemphigoid; Optic neuritis; Palindromic rheumatism (PR); PANDAS; Paraneoplastic cerebellar degeneration (POD); Paroxysmal nocturnal hemoglobinuria (PNH); Parry Romberg syndrome; Pars planitis (peripheral uveitis); Parsonnage-Turner syndrome; Pemphigus; Peripheral neuropathy; Perivenous encephalomyelitis; Pernicious anemia (PA); POEMS syndrome; Polyarteritis nodosa; Polyglandular syndromes type I, II, III; Polymyalgia rheumatica; Polymyositis; Postmyocardial infarction syndrome; Postpericardiotomy syndrome; Primary biliary cirrhosis; Primary sclerosing cholangitis; Progesterone dermatitis; Psoriasis; Psoriatic arthritis; Pure red cell aplasia (PRCA); Pyoderma gangrenosum, Raynaud's phenomenon; Reactive Arthritis; Reflex sympathetic dystrophy; Relapsing polychondritis; Restless legs syndrome (RLS); Retroperitoneal fibrosis; Rheumatic fever; Rheumatoid arthritis; Sarcoidosis; Schmidt syndrome; Scleritis; Scleroderma; Sjogren's syndrome; Sperm & testicular autoimmunity; Stiff person syndrome (SPS); Subacute bacterial endocarditis (SBE); Susac's syndrome; Sympathetic ophthalmia (SO); Takayasu's arteritis; Temporal arteritis/Giant cell arteritis; Thrombocytopenic purpura (TTP); Tolosa-Hunt syndrome (THS); Transverse myelitis; Type 1 diabetes; Ulcerative colitis (UC); Undifferentiated connective tissue disease (UCTD); Uveitis; Vasculitis; Vitiligo; Vogt-Koyanagi-Harada Disease; or Wegener's granulomatosis (or Granulomatosis with Polyangiitis (GPA)).

As used in this specification and the appended claims, the singular forms “a,” “an,” and “the” include plural referents, unless the context clearly dictates otherwise. The terms “a” (or “an”), as well as the terms “one or more,” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Unless defined otherwise, all technical and scientific terms used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology, P-S. Juo, (2d ed. 2002) can provide one of skill with general definitions of some terms used herein.

Units, prefixes, and symbols are denoted in their Système International de Unites (SI) accepted form. Numeric ranges are inclusive of the numbers defining the range. The headings provided herein are not limitations of the various aspects or embodiments of the invention, which can be had by reference to the specification as a whole. Accordingly, the terms defined immediately below are more fully defined by reference to the specification in its entirety.

Wherever embodiments are described with the language “comprising,” otherwise analogous embodiments described in terms of “consisting of” and/or “consisting essentially of” are included.

The following examples are provided to further illustrate the advantages and features of the present invention, but they are not intended to limit the scope of the invention. While the examples are typical of those that might be used, other procedures, methodologies, or techniques known to those skilled in the art may alternatively be used.

### Example I

### Generation of Modified Stem Cells

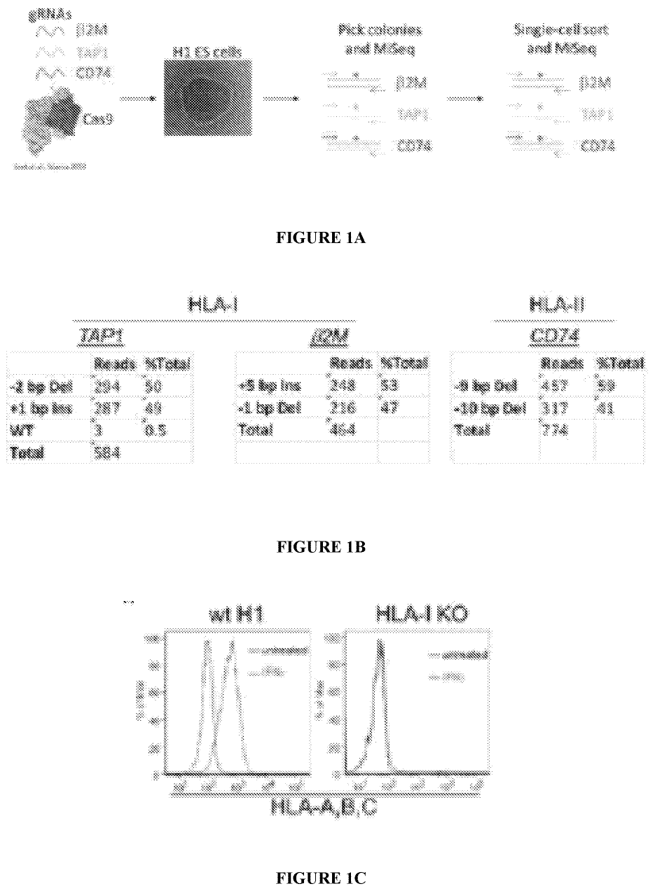

A sequential series of genetic variants of H1 human embryonic stem cells (hESC) was generated through CRISPR-based targeted mutations and lentiviral expression of immune evasion cassettes. Specifically, a workflow was optimized to generate targeted mutations in human ES cells. A Cas9-expression construct and up to 3 different gRNA-encoding vectors are co-transfected into H1 human ES cells. Individual colonies are manually picked and subjected to MiSeq™ analysis of targeted genes to identify clones carrying frameshift mutations introduced by non-homologous end joining errors. Candidate clones are then plated at exactly 1 cell/well by fluorescence-activated cell sorting (FACS), and MiSeq™ analysis is again performed to confirm the mutations and lack of mosaicism (FIG. 1A). Next, clones are expanded, karyotyped, and their differentiation potential is confirmed. Through CRISPR/Cas9-based targeted mutations, a karyotypically normal hES cell line has been generated that lacks HLA expression. One clone was identified carrying inactivating mutations in both alleles of β2m and TAP1 and in one allele of CD74 through MiSeq™ analysis of the targeted regions (FIG. 1). In this clone, Interferon-gamma-induced HLA-I expression was completely abrogated (FIG. 1C), and a normal karyotype was confirmed. Monocytes derived from this HLA-I-deficient line failed to stimulate allogeneic primary CD8+ T cell proliferation. This HLA-I-deficient line was subsequently re-targeted using CRISPR to ablate the remaining allele of CD74 and both alleles of CIITA, a transcription factor required for expression of HLA-II. This clone was also confirmed to possess a normal karyotype. As the absence of HLA-I may render target cells susceptible to NK cell-mediated cytolysis, the inventors also targeted the NKG2D ligands MICA and MICB through CRISPR/Cas9. RNA-seq analysis demonstrates that these are the only two NKG2D ligands expressed by pancreatic β cells. Sequencing of approximately 400 nucleofected clones revealed one line carrying frameshift mutations in all 4 alleles of MICA and MICB. This clone has been validated for normal karyotype and lack of MICA/MICB expression. HLA, MICA/B deficient hES cells will henceforth be referred to as HM-KO hES.

### Example II

### Humanized Mice Fail to Recapitulate Normal Immune Rejection Responses

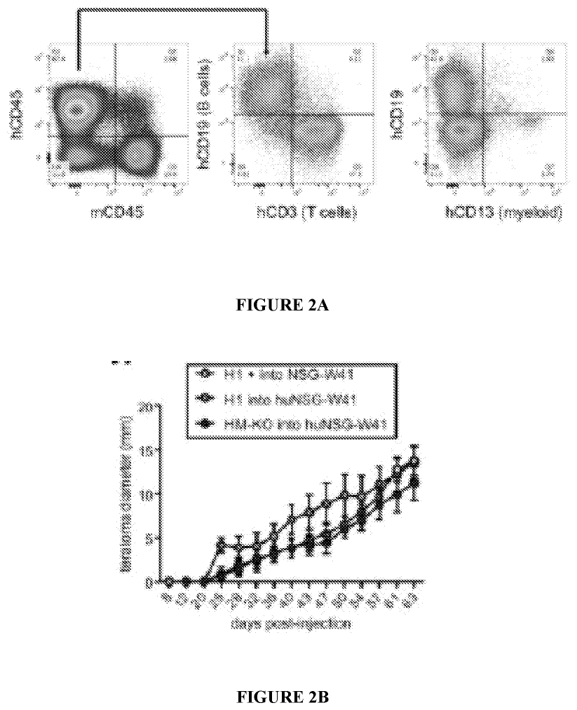

To test immune evasion by this line in vivo, a humanized mouse approach was used by transplanting 2×10⁵ cord blood CD34+ cells into unconditioned NSG W41 mice. B and T cell reconstitution in these mice was robust (FIG. 2A). Yet when these humanized mice were injected subcutaneously even with unmodified WT H1 hES cells (10⁶), teratoma growth was robust and comparable to that in control unhumanized NSG mice (FIG. 2B). Moreover, HM-KO cells grew identically to unmodified H1 cells in humanized NSG animals. Thus, these cord blood-humanized mice are incapable of rejecting xenogeneic teratomas and are not a reliable surrogate of normal human immune responses. The inability to reject even unmodified cells likely involves poor antibody responses, an absence of functional NK cells, and antigen-presenting cells that are HLA mismatched with thymically derived T cells.

### Example III

### Xenogeneic Immunocompetent Mice Reject HLA Deficient Grafts

More stringent assays were used to test the immunogenicity of cells in vivo. Xenogeneic responses are among the steepest known immune barriers to engraftment. Immune responses occur exceptionally rapidly due to a high frequency and potency of xenoreactive T cells and preformed antibodies that mediate acute xenorejection. It was reasoned that if the cells were to overcome xenoreactivity, confidence would be gained that these cells could also overcome alloreactivity and autoimmunity to β cells in T1D patients. HLA-I-KO, HLA-I/II-KO, and HM-KO cells were therefore transplanted into fully immunocompetent C57B16/J mice. No teratoma growth was observed in any recipient at any timepoint. Thus, HLA- and NKG2D ligand-deficiency is insufficient to cross xenogeneic barriers.

### Example IV

### Expression of Immune Evasion Genes in HLA Deficient Grafts Allows Teratoma Growth

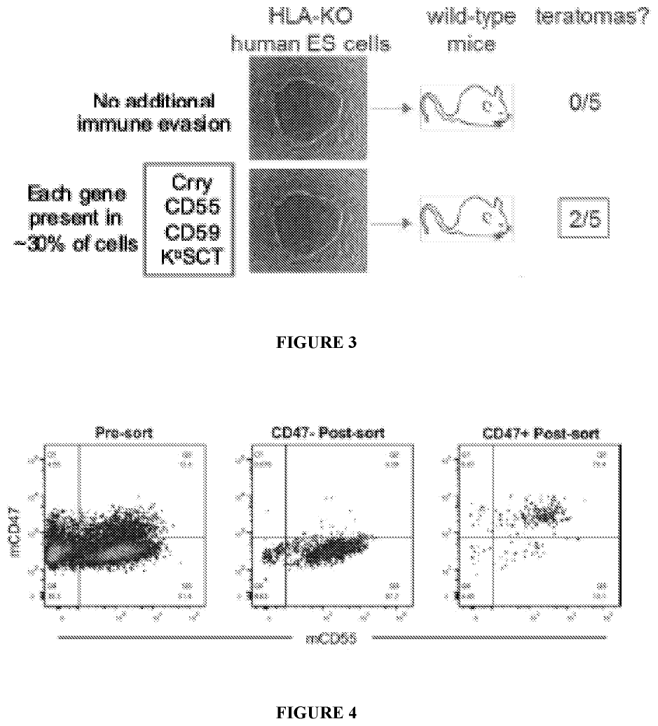

Aside from direct recognition by T cells, many other factors can mediate rejection. For example, CD4+ T cells can be primed indirectly by antigen-presenting cells that engulf foreign grafts. These indirectly primed T cells can then help B cells mount antibody responses against foreign targets. Graft-reactive antibodies, in turn, can elicit macrophage phagocytosis, NK cell activation, and complement deposition, all of which can lead to graft clearance. Genes were expressed that were predicted to alleviate each of these mechanisms of graft rejection. Individual lentiviral constructs were generated encoding a GFP marker and mouse orthologs of Crry, CD55, CD59, and K^{b}-single chain trimers. Crry, CD55, and CD59 inhibit complement activation and deposition⁶, and K^{b}-single chain trimers engage inhibitory Ly49C receptors on NK cells. HM-KO cells were transduced such that −30% were infected with any given lentivirus. This mixture of cells was then transplanted into fully immunocompetent C57B16/J mice. After 8 weeks, 2/5 mice showed small but clear teratomas (FIG. 3). Several mice showed transient growths during the monitoring period as well. In contrast, no growth was detectable at any timepoint when parental HM-KO cells were transplanted. These data suggested that a combination of immune evasion gene expression might allow HM-KO cells to avoid rejection and grow in xenogeneic recipients. Notably, CD47, which has been proposed as sufficient for allogeneic engraftment of HLA-I-deficient cells, was not included in these experiments and is therefore not necessary for teratoma growth.

### Example V

### Immune Evasion Gene Expression does not Impact Cell Differentiation

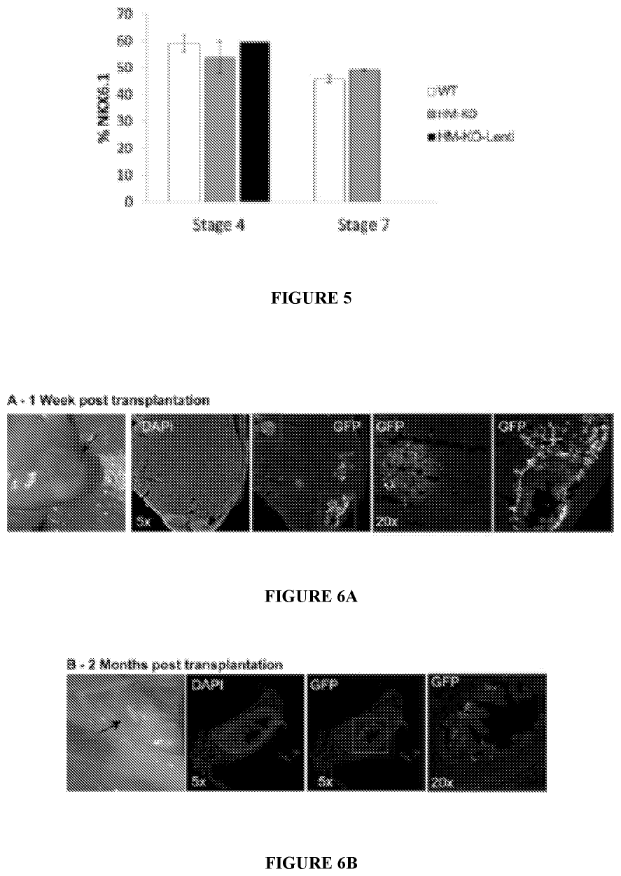

To extend upon these results, the inventors sorted these lentivirally-transduced HM-KO cells such that 100% expressed Crry, CD59, and K^{b}-single chain trimer. Approximately half of these cells also expressed CD55, and after additional transduction, 20% expressed CD47 (FIG. 4), which reduces macrophage phagocytosis. This mixture of cells is termed HM-KO-Lenti cells HM-KO and HM-KO-Lenti cells were differentiated into pancreatic β cells using automated procedures. It has been previously shown that HM-KO gene deletions did not affect the differentiation efficiency of hESC H1. Here, it is further shown that lentiviral overexpression of mouse evasion genes (HM-KO-Lenti) also did not affect differentiation efficiency (FIG. 5).

### Example VI

### Minimally Immunogenic Human Stem Cell Derived β Cells Persist in Wild Type (WT) Mice

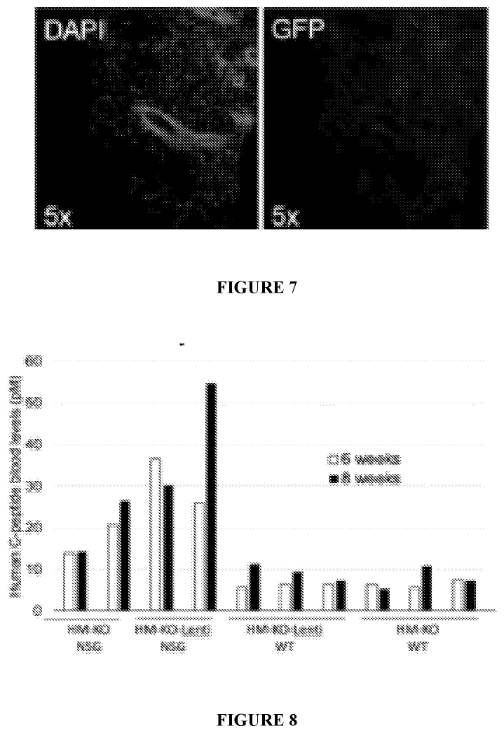

It was reasoned that transplanting human cells into fully immune competent WT mice would be a more relevant and stringent test to determine whether such cells could survive as grafts in the clinic. These xenogeneic barriers represent a considerable challenge, and probably exceed the actual barriers encountered to replace β cells in T1D patients. Therefore, it was hypothesized that if cells could cross this barrier, they could potentially survive in vivo when transplanted into patients with both autoimmune and allogeneic rejection barriers. Indeed, due to limitations of current humanized mouse models (see above), this xenogeneic barrier was deemed to be the only meaningful way the immune-evasive ability of these cells could be tested in vivo. In this experiment, 100 stem cell derived pseudo-islets derived from HM-KO and HM-KO-Lenti cells were transplanted subcutaneously into 6-8-week-old female mice (n=4). As a positive control, 100 pseudo-islets were also transplanted into immunodeficient NSG mice (n=2). To evaluate the survival of the pseudo-islets, one mouse per experimental group was sacrificed 1 week after transplantation. In the mouse transplanted with HM-KO-Lenti derived pseudo-islets, grafts were clearly visible at the transplantation site (FIG. 6A), and anti-GFP antibody stain confirmed their human origin through immunohistochemistry (IHC). Two months after transplantation, the remaining mice were sacrificed. None of the mice transplanted with HM-KO cells had visible grafts; however, 1 of 3 mice transplanted with HM-KO-Lenti cells had a small but clearly defined graft (FIG. 6B). The human stem cell origin of this tissue was also confirmed with GFP IHC (FIG. 6B). FIG. 7 shows staining of a neighboring mammary gland that is negative for GFP. These data demonstrate that expressing some combination of the immune evasion genes above allows persistent escape of xenorejection. The inventors are unaware of any prior literature that has ever reported such a result.

### Example VII

### Minimally Immunogenic Human Stem Cell-Derived β Cells are Functional In Vivo

NSG mice transplanted with HM-KO and HM-KO-Lenti cells had detectable human C-peptide in their blood, further demonstrating that the genetic modifications did not hamper cell differentiation (FIG. 8). WT mice transplanted with either type of ells did not show significant human C-peptide levels at the two relatively early timepoints tested. Confirmation that these grafts express insulin is being confirmed, as in their counterparts that were transplanted into NSG mice. Yet the most likely explanation for the absence of detectable human C-peptide in WT mice is that the number of pseudo-islets was clearly reduced relative to immunodeficient recipients of these cell grafts. Together, the data suggest that a certain combination of immune evasion genes allows grafts to persist in immunocompetent xenogeneic recipients while pseudo-islets lacking this ideal combination may still be rejected.

### Example VIII

### Redesigned Silencing-Resistant AAVS1-Targeting Constructs to Express Immune Evasion and Suicide Genes

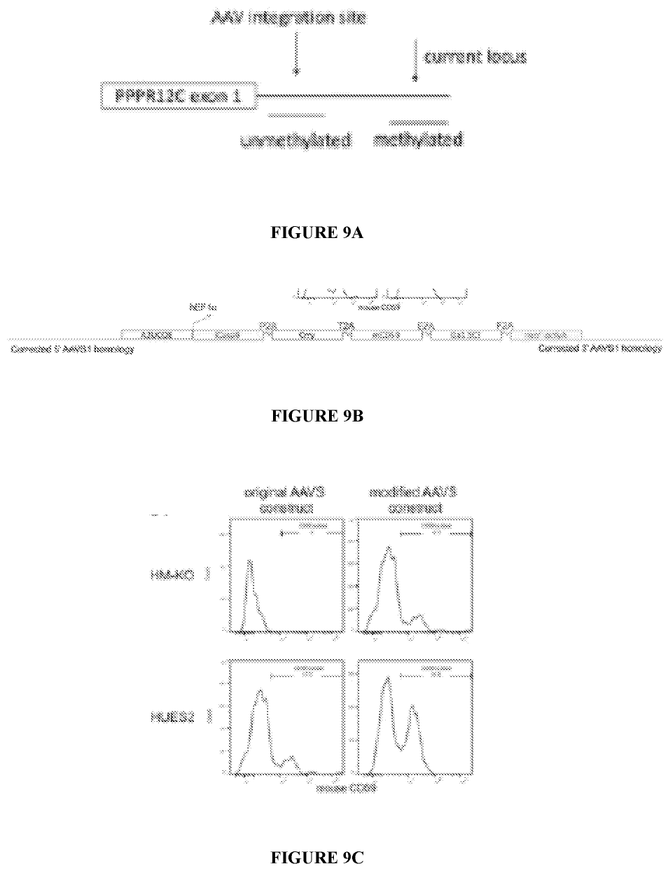

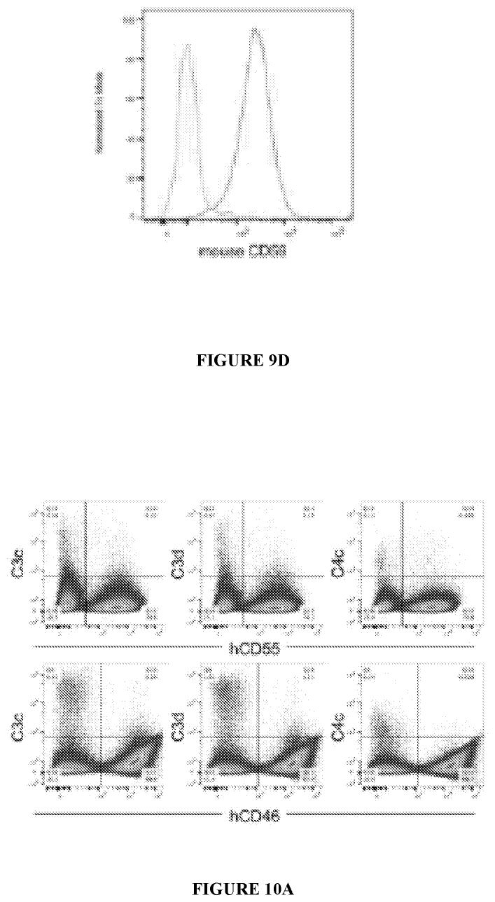

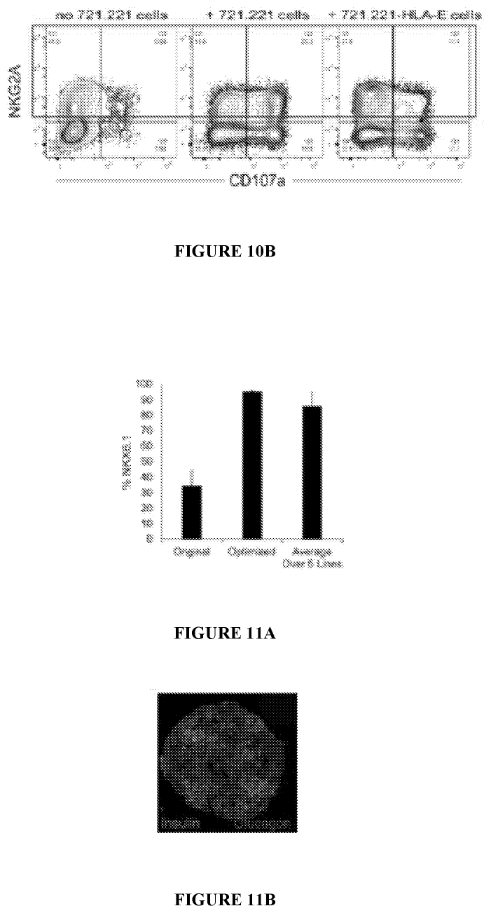

While the above lentiviral studies are useful to define essential combinations of immune evasion genes, this is not a clinically viable approach. Random integration of lentiviruses could activate oncogenes and/or silence expression, thereby leading to loss of immune evasion and graft loss. Inclusion of inducible suicide cassettes, such as mTK and iCasp9, would allow for pharmacological elimination of grafts if such unanticipated adverse events arise. Moreover, a defined locus to express the necessary immune evasion and suicide genes would avoid problems with random integration and silencing. Several studies have reported that the site of endogenous AAV integration, located in an intronic region of PPPR12C, is a ‘safe harbor’ for expression of exogenous genes in human pluripotent stem cells. However, it has been shown that all reported AAVS1 constructs and Cas9/gRNA systems target a region that can become highly methylated, instead of the endogenous AAV integration site, which is protected from silencing (FIG. 9A). Therefore, new immune evasion constructs were generated to target this more appropriate site (FIG. 9B). In these vectors, the inducible suicide genes mTK or iCasp9, which induce cell death when exposed to ganciclovir or AP1903, are linked to immune evasion genes and drug resistance cassettes through viral 2A sequences. In addition, the inventors have included an A2UCOE insulator element to minimize the chance of transcriptional silencing. HM-KO cells, as well as HUES2 cells, an alternate HES cell line with strong endoderm differentiation potential, have now been targeted with these constructs. The results show that a larger fraction of drug resistant hES cells express immune evasion genes when targeted with the new constructs compared to the older constructs (FIG. 9C). Even neomycin resistant HM-KO cells failed to detectably express any immune evasion genes when targeted with older AAVS constructs, but a fraction of these cells retained mCD59 expression when transfected with the newer construct (FIG. 9C). HUES2 cells, a separate hES cell line, retained expression of immune evasion genes better than HM-KO cells (FIG. 9C). Yet even in the HUES2 line, the newer AAVS targeting cassette led to better expression of immune evasion genes (FIG. 9C). The inventors selected drug-resistant cells, sorted and expanded clones expressing immune evasion cassettes, and confirmed that they maintained stable expression of the transgenes over several months (FIG. 9D). HM-KO cells stably expressing human homologs of these immune evasion genes have also been selected and expanded. When these AAVS constructs expressing human CD55, CD46, and HLAE (homolog of Qa1) single chain trimers were transfected into CHO or 721.221 cells, complement deposition and degranulation by NKG2A+ NK cells were markedly attenuated (FIGS. 10A and 10B), confirming the function of these targeting constructs.

### Example IX

### Optimization of Automated β Cell Differentiation Protocol

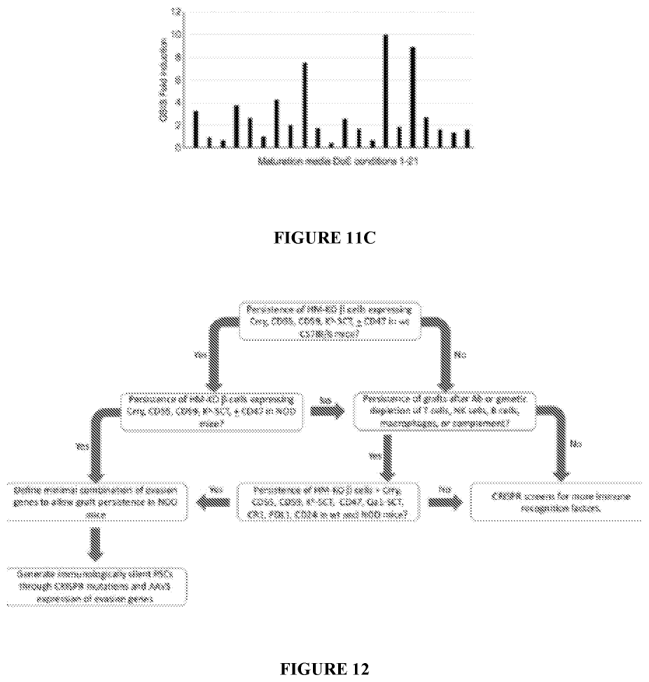

For this study, the same differentiation protocol was employed that was used previously to facilitate direct comparisons of data, but in the meantime, significant improvements have been made to the β cell differentiation protocol, resulting in over 90% NKX6.1 expression and an average of 84% efficiency over 5 cell lines (FIG. 11A). IHC staining of representative pseudo-islets showed significant insulin expression and isolated glucagon expression (FIG. 11B). Significant Design of Experiment (DoE) efforts have further identified maturation media conditions that result in pseudo-islets with robust glucose-stimulated insulin secretion (GSIS) responses after only 10 days of maturation (FIG. 11C). This will allow detailed functional characterization of genome-edited cell lines and comparisons to unmodified cells. Moving forward, these protocols will be applied.

### Example X

### Treatment of T1D Using Modified Stem Cells

Based on the results presented herein, the inventors seek to develop universal donor cells for diabetes cell replacement therapies. This will be accomplished by generating and confirming use of evasion gene constructs, performing preclinical proof of principle experiments in WT and NOD mice as well as generating a new HM-KO cell line that stably expresses selected evasion gene constructs through AAVS targeting (FIG. 12).

Aim 1. Demonstrate that Immune Evasion Gene Expression Prevents Rejection of Human Stem Cell-Derived β Cells in WT and NOD Mice.

This will be accomplished by transplanting cells that uniformly express immune evasion genes into WT mice. Remaining immune barriers will be defined via antibody depletion experiments. NOD mice will then be transplanted with cells that uniformly express immune evasion genes.

Aim 2. Define Minimal Combination of Genes to Prevent Rejection of Human Stem Cell-Derived Cells in NOD Mice.

This will be accomplished by defining functionally essential categories of immune evasion genes through in vivo selection assays. Minimal combinations of immune evasion genes will also be defined through limiting lentiviral infections and in vivo competition assays. A new HM-KO cell line will be generated that stably expresses a combination of mouse immune evasion and inducible suicide genes. Immunogenicity will be tested through in vitro assays of HM-KO cells that stably express human immune evasion genes.

Rationale.

T1D is caused by a complex autoimmune reaction. T1D is an autoimmune disorder in which T cells eliminate insulin-producing pancreatic β cells in the islets of Langerhans. Through a combination of human genetics, transplantation, cadaveric studies, and robust mouse models of T1D, much is now known regarding the mechanisms behind autoimmune destruction of β cells. Specific alleles of HLA-DQβ predispose to T1D, strongly implicating CD4+ T cells in disease onset. Mice carrying an analogous MHC II allele of I-A^{g7} also develop spontaneous T1D, presenting many of the same peptides as HLA-DQ and mimicking key aspects of human disease. In human T1D, many pancreatic lymph node T cells are reactive to insulin itself. In NOD mice, T1D is prevented by mutation of insulin such that the antigenic peptide cannot be presented on MHC II. The first insulin-reactive CD4+ T cells infiltrate the pancreas and draining lymph nodes to interact with a specialized macrophage population and cross-presenting dendritic cells that present antigenic insulin peptides. Once these CD4+ T cells become locally activated, the autoimmune response becomes progressively more complex. CD4+ T cell infiltrates are followed by self-reactive CD8+ T cells, which are accompanied by insulin-reactive B cells and antibodies. Though insulin-reactive cells predominate, the number of autoantigens recognized by CD4+ and CD8+ T cells begins to spread, eventually encompassing hundreds of self-peptides. These self-reactive lymphocytes and antibodies persist long after destruction of pancreatic β cells, such that islet transplants from even non-diabetic identical twins are rejected. Thus, the pre-existing immune response to β cells is exceptionally complex and represents a major barrier to pluripotent stem cell (PSC)-based replacement therapies.

Pluripotent stem cells are a scalable source of transplantable β cells. Although the standard of care for the control of T1D (through daily insulin injections) has been established, no cure has been developed to date. Landmark studies have proven that cadaveric donation of pancreatic islet transplantation restores β cell function and reverses T1D in recipients. Because of the shortage in pancreas organ donation, in addition to the side effects that immunosuppressive therapy carries, substantial efforts have been made to generate β cells from alternate and scalable sources. Directed differentiation of human PSCs represents the most advanced of these approaches as these cells can expand indefinitely in culture, thereby providing a reliable source of β cells that can be transplanted to a large population. Several robust protocols now exist to develop large numbers of β cells from human PSCs. Importantly, these cells have been shown to restore normal blood glucose levels in animal models of diabetes. Left unaddressed, however, are the autoimmune and allogeneic immune barriers to engraftment and persistence of transplanted β cells in T1D patients. Given that candidate patients for such procedures will have necessarily rejected their own β cells, strategies must be developed to allow pluripotent stem cell-derived replacement grafts to avoid similar immunological clearance. As systemic immunosuppressive therapy is unlikely to be acceptable to most T1D patients, an alternative approach is to genetically modify the graft to evade the host immune response. An added advantage to this approach would be the creation of a ‘universal’ donor cell line, dramatically reducing the costs of cell replacement therapies.

Unprecedented progress in overcoming T1D immune barriers. Recent studies have reported reducing the immunogenicity of human ES cells through genetic modifications. Yet these efforts have been limited to a relatively small number of immune recognition pathways primarily focused on HLA-I expression. These efforts are unlikely to cover the breadth of responses in T1D that pre-exist transplantation. One of these studies made an additional modification by expressing HLA-E, a non-polymorphic non-classical HLA-I molecule. HLA-E interacts with inhibitory NKG2A on NK cells, representing an appealing first approach. However, only approximately 20-50% of NK cells typically express NKG2A. Thus, expression of HLA-E is highly unlikely to overcome the additional immune barriers of HLA I-deficient cells. A second study reported that overexpression of CD47 in ES cells was sufficient to overcome allogeneic barriers in the context of HLA-deficient humanized mouse model. Though intriguing, the mechanism by which CD47 overexpression mediates immune evasion is unclear. Natural killer (NK) cell evasion was proposed, yet NK cells are unnecessary for graft rejection and do not express Sirpa, the ligand for CD47. A third study tested a larger breadth of molecules, including HLA-G and PD-L1; however, the resulting cells were only tested in humanized mouse models, which have been shown to be highly limited in their ability to reflect normal immune responses. Taken together, these efforts seem insufficient to define T1D immune barriers to pluripotent stem cell-based replacement therapies. Instead, a more comprehensive ablation of multiple aspects of the immune response, including direct T cell recognition, phagocytosis and indirect antigen presentation, antibody effector functions, and NK cell recognition, offers more promise. Indeed, the observations described herein that a subset of cells and grafts can survive in xenorecipients is unprecedented.

Genetic engineering of stem cells enables safe and sustainable surpassing of T1D immune barriers. As the preliminary data would predict, after overcoming both xenogeneic and autoimmune barriers in NOD mice, the next step will be to define the minimal essential components for overcoming these barriers. There are several reasons for this. First, excessive expression of immune evasion genes may predispose grafts to infections by opportunistic pathogens, or to tumorigenesis through lack of immune surveillance. Moreover, excessive expression of anti-phagocytic genes may limit the normal clearance of dying cells, leading to bystander inflammation and immunopathology. Second, if these non-immunogenic cells and transplantation strategies are to reach the clinic, defining the minimal combination of essential genes to express may ease the regulatory approval process. CRISPR genome editing, will be used to make targeted mutations as described above for H1 hES cells. In addition, whole genome sequencing will be performed between each round to ensure no deleterious mutations arise. Because lentiviruses can become silenced through passage and differentiation, and because these vectors can integrate in proto-oncogenes, immune evasion genes will be expressed at defined loci. The minimal combination of immune evasion genes, defined in Aim 2, will thus be expressed alongside inducible suicide genes in silencing-resistant AAVS1 targeting cassettes. These suicide genes might become important for eliminating grafts if unanticipated adverse events occur.

Research Design and Methods

Aim 1. Demonstrate that Immune Evasion Gene Expression Prevents Rejection of Human Stem Cell-Derived β Cells in WT and NOD Mice.

Transplant β Cells Uniformly Expressing Immune Evasion Genes into WT Mice.

The data herein suggests that immune evasion gene expression in HM-KO-Lenti cells can at least partially avoid xenograft rejection. As mentioned above, only half of the transplanted cells expressed CD55 and only 20% of these expressed CD47 (FIG. 4). Thus, the likely reason that the grafts persisted only partially is that only −10% of the input HM-KO-Lenti cells expressed all 5 immune evasion genes. Moreover, some degree of lentiviral silencing is inevitable during ES cell passaging and differentiation. The inventors have thus begun sorting HM-KO-Lenti cells for those expressing CD55 and CD47 in addition to Crry, CD59, and K^{b}-single chain trimer. These cells and control HM-KOs will be differentiated into β cells and transplanted in parallel into immunocompetent C57B16/J and immunodeficient NSG mice. Serum levels of human C-peptide will be quantified over the course of 8-12 weeks. At the final week, mice will be given a glucose tolerance test, sacrificed, and pseudo-islets will be sectioned. GFP and insulin expression in remaining grafts will be quantified and the infiltration of host-derived cells into the graft will be assessed with IHC. After each differentiation, HM-KO-Lenti and unmodified control pseudo-islets will be subjected to detailed functional and compositional evaluation to ensure the genetic modifications do not have adverse effects on β cell function.

Define Remaining Immune Barriers Via Antibody Depletion Experiments.

If fewer pseudo-islets and human C-peptide are observed in C57B16/J recipients relative to NSG recipients, the inventors will perform antibody depletion and genetic experiments to define remaining immune barriers. One day prior to transplantation with HM-KO-Lenti cells, C57B16/J recipients will be treated with depleting antibodies against CD4 and CD8 to remove T cells, NK1.1 antibodies to ablate NK cells, CD20 antibodies to deplete β cells, or CSF1 blocking antibodies to ablate macrophages and monocytes. In parallel, C3^{-/-} complement-deficient recipients will be transplanted with HM-KO-Lenti-derived β cells. As above, serum C-peptide levels will be measured over time, and persistence of pseudo-islets quantified. If T cell and/or CSF1 depletion is required to allow graft persistence, the inventors will further transduce HM-KO-Lenti cells with viruses encoding PDL1 and CD24. Aside from direct inhibition of T cells, PDL1 also prevents phagocytosis and antigen presentation to T cells by macrophages; CD24 exerts similar effects. If NK cells are required for graft rejection, the inventors will express Qa1-single chain trimer, which engages the inhibitory NKG2A receptor on NK cells. Moreover, the inventors will ablate the ULBP family of NKG2D ligands to further attenuate NK cell activation. Finally, if CD20 ablation and/or C3-deficiency allows graft acceptance, the inventors will express CR1 on HM-KO-Lenti cells. CR1 is an extremely potent inhibitor of complement activation, with greater efficiency and more rapid kinetics than Crry, CD55, and CD59. Upon expression of additional immune evasion genes as guided by these antibody depletion experiments, β cell transplantation experiments will be performed in C57B16/J mice as above.

Transplant β Cells Uniformly Expressing Immune Evasion Genes into NOD Mice.

Once engraftment in immunocompetent mice is maximized, the inventors will raise the biological stringency of the assays. In a clinical situation, β cell grafts would be given only to those with active T1D. In this setting, preformed β cell-reactive antibodies, memory T cells, and associated inflammatory conditions would pre-exist the graft. In mice, this situation is best mimicked in NOD mice. Autoantigens such as insulin itself are highly conserved between mice and humans, making it likely that antibodies in NOD mice would cross-react with the human β cell graft. Moreover, indirect presentation of graft-derived antigens could lead to further T cell activation, inflammation, and graft loss. To test these possibilities, the inventors will use the optimal combination of HM-KO-Lenti cells defined above to generate β cells. These cells will be transplanted into 8-week-old female NOD mice obtained from Jackson Labs. These mice reliably develop T1D by 30 weeks of age, with pancreatic immune infiltrates and insulin antibodies apparent as early as 4 weeks. After transplantation, serum human C-peptide and glucose levels will be monitored. It is expected that HM-KO-Lenti-derived grafts will persist, produce human insulin, and prevent T1D.

Expected Results.

It is expected that HM-KO-derived β cells expressing CR1, CD55, CD47, CD59, Crry, Qa1- and K^{b}-single chain trimers, PDL1, and CD24 will efficiently engraft and persist in C57B16/J and NOD mice. It is expected that these grafts will be resistant to autoimmune rejection in NOD mice; thus, recipients of these grafts will not manifest with T1D.

Aim 2. Define Minimal Combinations of Genes to Prevent Rejection of Human Stem Cell-Derived β Cells in NOD Mice.

Define Functionally Essential Categories of Immune Evasion Genes Through In Vivo Selection Assays.

The inventors will undertake a systematic approach to identify and exclude non-essential immune evasion genes. The inventors will first categorize genes into functionally distinct categories as shown in Table 1. H1 hES cells, HLA-I-deficient cells, HLA-I/II-deficient cells, and HM-KO cells will be transduced with specific combinations of these genes such that one functional category is excluded. For example, HM-KO cells will be transduced with all aforementioned immune evasion genes except CD47, PDL1, and CD24 to determine the importance of preventing phagocytosis. Other pluripotent stem cells will be transduced with all immune evasion genes except Qa1- and K^{b}-single chain trimers to test the importance of NK cell-mediated rejection. These pools of cells will be mixed together and differentiated into β cells. A small sample of pseudo-islets will be dissociated and tested by flow cytometry for the relative contributions of each cellular pool. The remaining pseudo-islets will be transplanted into NOD mice. At 8 weeks post-transplant, grafts will be recovered and the representations of each lentiviral pool of cells will be quantified relative to the pre-transplant frequencies. Through these assays, categories of genes that are essential for graft persistence and immune evasion will be defined.

TABLE 1
Immune evasion pathways and genes.

| Pathway/Cell Type Inhibited | Genes |
|---|---|
| NK cells | Qa1/HLA-E single chain trimer, |
| | K^{b}/HLA-G single chain trimer |
| complement and antibodies | CR1, CD46/Crry, CD55, CD59 |
| phagocytosis and T cell priming | CD47, PDL1, CD24 |

Define Minimal Combinations of Immune Evasion Genes Through Limiting Lentiviral Infections and In Vivo Competition Assays.

Once the inventors have defined functional categories of genes that are essential for evading xenorejection, they will define the essential genes in that category. For example, if it is found that complement evasion is essential for engraftment and persistence, the inventors will first lentivirally express all immune evasion genes except CR1, Crry, CD55 and CD59. These cells will then be transduced with each individual complement evasion gene such that −30% are infected. This pool of cells will be differentiated to cells, analyzed by flow cytometry for expression of these complement evasion genes, and transplanted. As above, the inventors will quantify enrichment and loss of cells that express these complement evasion factors. For example, if cells expressing CR1 are enriched post-transplant relative to input cells, but those expressing Crry are not, it will be concluded that CR1 is essential but Crry is not. Through this iterative process, the inventors will define the minimal combination of immune evasion genes to express and HLA/NKG2D ligand genes to mutate to allow graft persistence.

Generate New HM-KO Cell Line that Stably Expresses Optimal Combination of Mouse Immune Evasion and Inducible Suicide Genes Through AAVS Targeting.

As discussed above, lentiviral overexpression is not a clinically viable solution to express immune evasion or suicide genes. Instead, using the information from these lentiviral experiments, the inventors will use CRISPR genome editing to generate AAVS1 targeting constructs as in FIG. 9 that express the minimal combination of mouse immune evasion genes, a neomycin resistance cassette, and an inducible suicide gene of either HSV thymidine kinase or iCasp9, all linked together with ribosome-skipping viral 2A sequences. As in FIG. 9, these targeting constructs will be transfected into HM-KO cells along with Cas9 and a gRNA targeting the proper AAVS locus. Neomycin-resistant cells will be selected, and cells stably expressing mouse immune evasion genes will be sorted clonally and expanded. After karyotyping and exome sequencing to confirm the absence of oncogenic mutations, the inventors will differentiate these pluripotent stem cells into cells and transplant into 8-week-old NOD female mice. Serum levels of glucose and human C-peptide will be measured over time to confirm that AAVS-targeted cells behave similarly to lentivirally transduced cells.

Test Immunogenicity Through In Vitro Assays of HM-KO Cells that Stably Expresses Optimal Combination of Human Immune Evasion Genes.

To this point, all of the assays have focused on mouse xenotransplantation as an in vivo measure of immune evasion. Yet the barriers could be different in the actual clinical setting of allotransplantation. This is difficult to model completely, but a set of relevant in vitro assays can be employed to gain further confidence in the strategy. HM-KO cells expressing human homologs of essential immune evasion genes will be generated through AAVS1 targeting. These cells will be differentiated into cells and used for in vitro immune recognition assays.

Expected Results.

It is expected that 1-2 genes in each functional category of immune evasion will be necessary for graft persistence. The inventors expect that these genes and inducible suicide cassettes will be stably expressed through AAVS1 targeting, which will prevent immune recognition both in vivo and in vitro. Further, it is expected that in vitro measures of immune recognition will be sharply diminished in HM-KO cells expressing human immune evasion genes.

### Example XI

### Ability of CR1 and/or CD24 to Improve Immune Evasion in Stem Cells and a Variety of Derivative Tissues

Goal

Test the ability of CR1 and/or CD24 to improve immune evasion in stem cells and a variety of derivative tissues (including but not limited to: microglia; retinal pigmented epithelia; astrocytes; oligodendrocytes; hepatocytes; podocytes; keratinocytes; cardiomyocytes; dopaminergic neurons; cortical neurons; sensory neurons; NGN2-directed neurons; interneurons; basal forebrain cholinergic neurons; pancreatic beta cells; neural stem cells; natural killer cells; regulatory T cells; lung cell lineages; kidney cell lineages; blood cell lineages), with reduced HLA-I and HLA-II as well as increased expression of CD47, CD55, CD46, CD59 and HLA-E-single chain trimer.

Experiment 1

Expression of CR1 and/or CD24 will be increased in stem cells and variety of derivative tissues (including but not limited to: microglia; retinal pigmented epithelia; astrocytes; oligodendrocytes; hepatocytes; podocytes; keratinocytes; cardiomyocytes; dopaminergic neurons; cortical neurons; sensory neurons; NGN2-directed neurons; interneurons; basal forebrain cholinergic neurons; pancreatic beta cells; neural stem cells; natural killer cells; regulatory T cells; lung cell lineages; kidney cell lineages; blood cell lineages), with reduced HLA-I and HLA-II as well as increased expression of CD47, CD55, CD46, CD59 and HLA-E-single chain trimer. In vitro assays will be performed to assess the ability of CR1 to reduce complement deposition through the classical antibody-dependent pathway, and for CD24 to reduce phagocytosis of antibody-coated cells by macrophages.

Experiment 2

Expression of CR1 and/or CD24 will be increased in stem cells and variety of derivative tissues (including but not limited to: microglia; retinal pigmented epithelia; astrocytes; oligodendrocytes; hepatocytes; podocytes; keratinocytes; cardiomyocytes; dopaminergic neurons; cortical neurons; sensory neurons; NGN2-directed neurons; interneurons; basal forebrain cholinergic neurons; pancreatic beta cells; neural stem cells; natural killer cells; regulatory T cells; lung cell lineages; kidney cell lineages; blood cell lineages), with reduced HLA-I and HLA-II as well as increased expression of mouse homologs of CD47, CD55, CD46 (Crry), CD59 and HLA-E-single chain trimer (Qa1). Fort mouse experiments, HLA-G single chain trimer (Kb-single chain trimer) will be included. The survival of these cells after transplantation into immune competent WT C57BL6 mice and effect CR1 and/or CD24 has on survival and function of the respective tissues will be assessed.

Expected Outcome

CR1 and/or CD24 will increase the immune evasive capabilities and thus survival of the xeno-transplanted tissues. Repeat for in vitro assays.

### Example XII

### Ability of CR1 and/or CD24 to Replace Immune Factors

Goal

Test the ability of CR1 and/or CD24 to replace any of the following factors, CD47, CD55, CD46, CD59 and HLA-E-single chain trimer, in stem cells and a variety of derivative tissues (including but not limited to: microglia; retinal pigmented epithelia; astrocytes; oligodendrocytes; hepatocytes; podocytes; keratinocytes; cardiomyocytes; dopaminergic neurons; cortical neurons; sensory neurons; NGN2-directed neurons; interneurons; basal forebrain cholinergic neurons; pancreatic beta cells; neural stem cells; natural killer cells; regulatory T cells; lung cell lineages; kidney cell lineages; blood cell lineages), with reduced HLA-I and HLA-II expression while maintaining or improving survival and function after in vivo transplantation into immune competent WT C57BL6 mice.

Experiment 1

Expression of CD24 will be increased in stem cells and a variety of derivative tissues (including but not limited to: microglia; retinal pigmented epithelia; astrocytes; oligodendrocytes; hepatocytes; podocytes; keratinocytes; cardiomyocytes; dopaminergic neurons; cortical neurons; sensory neurons; NGN2-directed neurons; interneurons; basal forebrain cholinergic neurons; pancreatic beta cells; neural stem cells; natural killer cells; regulatory T cells; lung cell lineages; kidney cell lineages; blood cell lineages), with reduced HLA-I and HLA-II as well as increased expression of mouse homologs of CD55, CD46 (Crry), CD59 and HLA-E-single chain trimer (Qa1) (no CD47—phagocytosis), and the survival of these cells after transplantation into immune competent WT C57BL6 mice and effect CR1 and/or CD24 has on survival and function of the respective tissues will be assessed.

Experiment 2

Expression of CR1 will be increased in stem cells and a variety of derivative tissues (including but not limited to: microglia; retinal pigmented epithelia; astrocytes; oligodendrocytes; hepatocytes; podocytes; keratinocytes; cardiomyocytes; dopaminergic neurons; cortical neurons; sensory neurons; NGN2-directed neurons; interneurons; basal forebrain cholinergic neurons; pancreatic beta cells; neural stem cells; natural killer cells; regulatory T cells; lung cell lineages; kidney cell lineages; blood cell lineages), with reduced HLA-I and HLAII as well as increased expression of mouse homologs of CD47, CD46 (Crry), and HLA-E-single chain trimer (Qa1) (no CD55, CD59—complements), and the survival of these cells after transplantation into immune competent WT C57BL6 mice and effect CR1 and/or CD24 has on survival and function of the respective tissues will be assessed.

Expected Outcome

CR1 and/or CD24 will be able to induce immune evasion in stem cells and a variety of derivative tissues (including but not limited to: microglia; retinal pigmented epithelia; astrocytes; oligodendrocytes; hepatocytes; podocytes; keratinocytes; cardiomyocytes; dopaminergic neurons; cortical neurons; sensory neurons; NGN2-directed neurons; interneurons; basal forebrain cholinergic neurons; pancreatic beta cells; neural stem cells; natural killer cells; regulatory T cells; lung cell lineages; kidney cell lineages; blood cell lineages), in the absence of different combinations of these factors CD47, CD55, CD46, CD59 and HLA-E-single chain trimer. In particular, the inventors especially expect CR1 to be able to replace the factors CD55, CD46, and/or CD59 and CD24 to replace CD47. Repeat for in vitro assays.

### Example XIII

### Ability of CR1 and/or CD24 to Replace Immune Factors

Goal

Test the ability of CR1 and/or CD24 to replace any of the following factors, CD47, CD55, CD46, CD59 and HLA-E-single chain trimer, in stem cells and a variety of derivative tissues (including but not limited to: microglia; retinal pigmented epithelia; astrocytes; oligodendrocytes; hepatocytes; podocytes; keratinocytes; cardiomyocytes; dopaminergic neurons; cortical neurons; sensory neurons; NGN2-directed neurons; interneurons; basal forebrain cholinergic neurons; pancreatic beta cells; neural stem cells; natural killer cells; regulatory T cells; lung cell lineages; kidney cell lineages; blood cell lineages), with reduced HLA-I and HLA-II expression while maintaining or improving survival and function after in vitro complement deposition and phagocytosis assays.

Experiment 1

Expression of CD24 will be increased in stem cells and a variety of derivative tissues (including but not limited to: microglia; retinal pigmented epithelia; astrocytes; oligodendrocytes; hepatocytes; podocytes; keratinocytes; cardiomyocytes; dopaminergic neurons; cortical neurons; sensory neurons; NGN2-directed neurons; interneurons; basal forebrain cholinergic neurons; pancreatic beta cells; neural stem cells; natural killer cells; regulatory T cells; lung cell lineages; kidney cell lineages; blood cell lineages), with reduced HLA-I and HLA-II as well as increased expression of mouse homologs of CD55, CD46 (Crry), CD59 and HLA-E-single chain trimer (Qa1) (no CD47—phagocytosis). In vitro assays will be performed to assess the ability of CD24 to reduce phagocytosis of antibody-coated cells by macrophages.

Experiment 2

Expression of CR1 will be increased in stem cells and a variety of derivative tissues (including but not limited to: microglia; retinal pigmented epithelia; astrocytes; oligodendrocytes; hepatocytes; podocytes; keratinocytes; cardiomyocytes; dopaminergic neurons; cortical neurons; sensory neurons; NGN2-directed neurons; interneurons; basal forebrain cholinergic neurons; pancreatic beta cells; neural stem cells; natural killer cells; regulatory T cells; lung cell lineages; kidney cell lineages; blood cell lineages), with reduced HLA-I and HLA-II as well as increased expression of mouse homologs of CD47, CD46 (Crry), and HLA-E-single chain trimer (Qa1) (no CD55, CD59—complements). In vitro assays will be performed to assess the ability of CR1 to reduce complement deposition through the classical antibody-dependent pathway.

Expected Outcome

CR1 and/or CD24 will be able to induce immune evasion in stem cells and a variety of derivative tissues (including but not limited to: microglia; retinal pigmented epithelia; astrocytes; oligodendrocytes; hepatocytes; podocytes; keratinocytes; cardiomyocytes; dopaminergic neurons; cortical neurons; sensory neurons; NGN2-directed neurons; interneurons; basal forebrain cholinergic neurons; pancreatic beta cells; neural stem cells; natural killer cells; regulatory T cells; lung cell lineages; kidney cell lineages; blood cell lineages), in the absence of different combinations of these factors CD47, CD55, CD46, CD59 and HLA-E-single chain trimer. In particular, the inventors expect CR1 to be able to replace the factors CD55, CD46, and/or CD59 and CD24 to replace CD47. Repeat for in vitro assays.

Although the invention has been described with reference to the above examples, it will be understood that modifications and variations are encompassed within the spirit and scope of the invention. Accordingly, the invention is limited only by the following claims.

What is claimed is:

1. A method of generating a stem cell (SC) comprising:
a) modifying a SC to reduce expression relative to a wild-type SC of HLA-I, HLA-II, or a combination thereof; and
b) introducing exogenous constructs to express immune evasion genes comprising CR1 and/or CD24 and optionally one or more of CD47, CD55, CD46, CD59 and HLA-E-single chain trimer.

2. The method of claim 1, wherein the immune evasion genes comprise CR1 and CD24.

3. The method of claim 1, wherein the immune evasion genes comprise CR1, CD24 and one or more of CD46, CD47, CD55, CD59 and HLA-E-single chain trimer.

4. The method of claim 1, wherein the immune evasion genes comprise CR1, CD24, CD47, CD55, CD46, CD59 and optionally HLA-E-single chain trimer.

5. The method of and of claims 1-4, further comprising introducing exogenous constructs to express one or more of PDL1 and HLA-G-single chain trimer.

6. The method of claim 1, wherein expression of HLA-I is reduced by abrogating expression of TAP1 or β2M.

7. The method of claim 1, wherein expression of HLA-II is reduced by abrogating expression of CD74 and CIITA.

8. The method of claim 1, wherein modifying comprises genome editing using CRISPR/Cas9 targeted mutation.

9. The method of claim 1, wherein introducing exogenous constructs is performed by lentiviral transduction.

10. The method of claim 1, wherein introducing exogenous constructs is performed using an adeno-associated virus (AAV) construct.

11. The method of claim 10, wherein the AAV construct is a modified AAVS construct that targets a site of endogenous AAV integration located in an intronic region of PPPR12C.

12. The method of any of claims 1-11, further comprising differentiating the SC to a β cell.

13. A stem cell (SC) produced by the method of any of claims 1-11.

14. The SC of claim 13, wherein:
(i) expression of HLA-I and HLA-II is abrogated; and
(ii) the SC expresses CR1 and/or CD24 and optionally one or more of CD47, CD55, CD46, CD59 and HLA-E-single chain trimer.

15. The SC of claim 14, wherein the SC further expresses PDL1 and HLA-G-single chain trimer.

16. The SC of any of claims 13-15, wherein the SC is a mouse or human SC.

17. The SC of claim 16, wherein the SC is an embryonic SC or induced pluripotent SC.

18. A method of treating a disease or disorder in a subject in need thereof with a SC of any of claims 13-17, or progeny of a SC of any of claims 13-17.

19. The method of claim 18, wherein the disease or disorder is an autoimmune disease or a neurodegenerative disease.

20. The method of claim 18, wherein the disease or disorder is cancer.

21. The method of claim 18, wherein the disease or disorder is type 1 diabetes.

22. A β cell produced by the method of claim 12.

23. A method of treating type 1 diabetes (T1D) in a subject, comprising administering a β cell of claim 22 to the subject, thereby treating T1D in the subject.

24. A stem cell (SC), wherein:
(i) expression of HLA-I and HLA-II is abrogated; and
(ii) the SC is genetically modified to express CR1 and/or CD24 and optionally one or more of CD47, CD55, CD46, CD59 and HLA-E-single chain trimer.

25. The SC of claim 24, wherein the SC is further genetically modified to express PDL1 and HLA-G-single chain trimer.

26. The SC of any of claims 24-25, wherein the SC is a mouse or human SC.

27. The SC of claim 26, wherein the SC is an embryonic SC or induced pluripotent SC.

28. A cell line derived from the SC of any of claims 24-27.

29. The method of any of claims 1-11, further comprising differentiating the SC to produce a differentiated cell or tissue.

30. The method of claim 29, wherein the cell or tissue is selected from the group consisting of microglia, retinal pigmented epithelia, astrocytes, oligodendrocytes, hepatocytes, podocytes, keratinocytes, cardiomyocytes, dopaminergic neurons, cortical neurons, sensory neurons, NGN2-directed neurons, interneurons, basal forebrain cholinergic neurons, pancreatic beta cells, neural stem cells, natural killer cells, regulatory T cells, lung cell lineages, kidney cell lineages and blood cell lineages.

31. A cell or tissue generated by the method of claim 29.

32. The cell or tissue of claim 31, wherein the cell or tissue is selected from the group consisting of microglia, retinal pigmented epithelia, astrocytes, oligodendrocytes, hepatocytes, podocytes, keratinocytes, cardiomyocytes, dopaminergic neurons, cortical neurons, sensory neurons, NGN2-directed neurons, interneurons, basal forebrain cholinergic neurons, pancreatic beta cells, neural stem cells, natural killer cells, regulatory T cells, lung cell lineages, kidney cell lineages and blood cell lineages.
